(19) United States
(12) Patent Application Publication
(10) Pub. No.: US 2011/0077222 A1
(43) Pub. Date: Mar. 31, 2011

(54) Sustained-Release Opiate and Opiate Derivative Compositions

(75) Inventors: Carl J. Schaefer, Crestwood, MO (US); Gary L. Cantrell, Troy, IL (US)

(73) Assignee: Mallinckrodt Inc., Hazelwood, MO (US)

(21) Appl. No.: 12/894,203

(22) Filed: Sep. 30, 2010

Related U.S. Application Data
(60) Provisional application No. 61/247,076, filed on Sep. 30, 2009.

(51) Int. Cl. A61K 31/485; C07F 9/09; A61P 25/04

(52) U.S. Cl. 514/81; 546/23

(57) ABSTRACT

The present invention provides sustained-release opiate compositions. In particular, the present invention provides sustained-release opiate compositions that include an opiate attached to a blood albumin binder. The present invention also relates to methods of administering an opiate with a sustained release pharmacokinetic profile.

### CROSS REFERENCE TO RELATED APPLICATIONS

This application claims the benefit of U.S. Provisional Application No. 61/247,076 filed Sep. 30, 2009, which is incorporated herein in its entirety.

### FIELD OF THE INVENTION

The present invention generally relates to sustained-release opiate compositions. In particular, the present invention relates to sustained-release opiate compositions that include an opiate attached to a blood albumin binder.

### BACKGROUND OF THE INVENTION

Opiates are highly effective and widely used narcotic compounds. Although these compounds have a powerful and essentially immediate effect, the benefits of opiate administration are somewhat limited by relatively short half-life of opiates due to rapid clearance by the hepatic and urinary systems.

Existing sustained-release opiate formulations entail administering the opiate active compound as an oral composition that includes the opiate active compounds coated with a degradable coating that releases the opiate active compound with a specified release profile. However, these formulations are relatively expensive to produce and are particularly vulnerable to tampering, posing the danger of overdose. Further, the release profile of oral sustained release compounds are relatively unpredictable due to wide variation in the chemical conditions in the stomach due to the ingestion of food, alcohol, or other pharmaceutically active compounds.

Although the opiate may be released in a sustained-release profile using an orally administered composition, the opiate is still rapidly cleared from circulation upon release. Thus the sustained-release period is limited to the amount of time that the oral composition is resident in a region of the digestive tract possessing suitable conditions for the release and absorption of the opiate active compounds.

The half-life of any active compound in circulation is affected by the affinity of the compound for long-lived circulating proteins such as blood albumin or blood cell surface proteins. Recent sustained-release formulations have attempted to exploit this phenomenon by coupling active compounds with antibodies, antibody fragments, and other peptides that have a binding affinity for blood albumin or other circulating proteins. In addition to extending the half-life of active compounds, the chemical properties of the bloodstream such as pH are actively maintained at constant levels, resulting in a more reliable release profile than oral sustained release compositions. To date, this approach has been limited to active compounds that are small peptides.

A need exists in the art for a sustained-release opiate composition that incorporates a circulating protein binding compound to extend the halt-life of the opiate in circulation and to provide a reliable pharmacokinetic release profile. However, the efficacy of opiates and opiate derivatives are notoriously sensitive to small changes to their chemical structures. A need exists in the art for a sustained-release formulation that incorporates a circulating protein binding compound without compromising the efficacy of the opiate active compound.

### SUMMARY OF THE INVENTION

One aspect of the present invention encompasses a sustained-release opiate composition that includes an opiate, a non-peptide blood albumin binder, and a connecting bridge attached to the opiate and to the non-peptide blood albumin binder. Without being bound to any particular theory, after administering the composition to a patient, the non-peptide blood albumin binder non-covalently binds to circulating albumin protein in the bloodstream of the patient. The opiate and binding bridge, which are attached to the non-peptide blood albumin binder, are similarly bound to the circulating albumin protein. In this bound state, the opiate is rendered inactive until released into the bloodstream. The bonds that attach the opiate to the binding bridge are cleaved as a function of time, and the opiate is released into the bloodstream to exert its therapeutic effect. It has been discovered surprisingly that an opiate released in this manner exhibits essentially the same efficacy as the same opiate administered in isolation.

Without being bound to any particular theory, the elapsed time at which the opiate is released is influenced by a number of factors including the chemical properties of the binding bridge and the opiate, as well as the chemical properties of the patient's bloodstream, such as pH. Because blood pH is actively maintained at a constant value, the pharmacokinetic release profile is highly reliable. In another aspect, the sustained-release opiate composition includes an opiate, a non-peptide blood albumin binder such as diphenylcyclohexanol, and a connecting bridge such as phosphate diester. Yet another aspect provides a sustained-release opiate composition that includes an opiate chosen from oxycodone, oxymorphone, hydrocodone, hydromorphone, naloxone, nalbuphine, nalmefene, buprenorphine, and naltrexone, a non-peptide blood albumin binder such as diphenylcyclohexanol, and a connecting bridge such as phosphate diester.

An additional aspect provides a sustained-release opiate composition that includes a first compound and a second compound. The first compound includes a first opiate, a first non-peptide blood albumin binder, and a first connecting bridge attached to the first opiate and to the first non-peptide blood albumin binder. The second compound includes a second opiate, a second non-peptide blood albumin binder, and a second connecting bridge attached to the second opiate and to the second non-peptide blood albumin binder.

Another additional aspect provides a sustained-release opiate composition that includes a first opiate, a second opiate, a non-peptide blood albumin binder, and a connecting bridge attached to the first opiate, to the second opiate, and to the non-peptide blood albumin binder.

Still another aspect provides a method of administering an opiate to a human subject with a sustained release pharmacokinetic profile relative to a pharmacokinetic profile of the opiate administered in isolation. In this aspect, the method includes providing a sustained-release opiate composition that includes an opiate, a non-peptide blood albumin binder, and a connecting bridge attached to the opiate and to the non-peptide blood albumin binder. The method further includes administering the sustained-release opiate composition to the human subject.

Other features and iterations of the invention are described in more detail below.

### DETAILED DESCRIPTION

The present invention provides sustained-release opiate compositions and methods of administering the compositions to a human patient. In particular, the present invention provides sustained-release opiate compositions that include an opiate attached to a blood albumin binder using a connecting bridge. Upon administration, the blood albumin binder component of the composition non-covalently binds to circulating blood albumin. The connecting bridge is cleaved as a function of time, thereby releasing the opiate into circulation, where it exerts its therapeutic effect. It has been discovered that administering the opiate attached to a blood albumin binder significantly extends the half-life of the opiate in the blood without significantly degrading the therapeutic efficacy of the opiate.

Without being bound to any particular theory, when bound to blood albumin, the sustained-release opiate composition is protected from processes of hepatic and nephritic elimination that rapidly remove unprotected opiates from the bloodstream. In addition, when the sustained-release opiate composition is bound to blood albumin, the opiate component is rendered inactive until the connecting bridge is hydrolyzed, thereby releasing the opiate in its active form into the bloodstream. Thus, the blood albumin functions as a pool of unreleased opiate that circulates in relatively close proximity to target opioid receptors. Unlike previous sustained-release formulations that rely on the degradation of a coating layer in the digestive system to extend the release of active compounds into the circulatory system, embodiments of the sustained-release opiate composition release opiate directly into circulation.

The sustained release profile of the opiate composition may be specified by administering a composition that includes a mixture of compounds in which each compound includes the opiate attached to the blood albumin binders using one of at least two different connecting bridges. Because each of the different connecting bridges may be cleaved in the bloodstream at different times, the release profile of the combined compounds is different from the release profile of any of the single compounds administered in isolation. Alternatively, the sustained release pharmacokinetic profile of the opiate composition may be specified by administering a composition in which two or more opiates are attached to each blood albumin binder using a connecting bridge. In other embodiments, a sustained release composition may include two or more different opiate compounds that are attached to the same albumin binding bridge.

The present invention further provides a method of administering an opiate in a sustained-release pharmacokinetic profile that includes administering a sustained-release composition that includes an opiate attached to a blood albumin binder using a connecting bridge. Because the sustained-release opiate composition is inactive when bound to blood albumin, a higher dosage may be administered relative to the dosage recommended for the opiate administered in isolation. Thus, the sustained-release opiate composition releases an effective amount of opiate into the bloodstream with a more uniform concentration over time and for a more sustained period of time than the opiate administered in isolation.

The sustained-release opiate compositions, as well as the opiates, blood albumin binders, and connecting bridges, are described in detail below.

### (I) Opiates

The opiate included in the embodiments of the sustained-release opiate compositions may be selected from opium, natural opium derivatives, semi-synthetic opium derivatives, and synthetic opium derivatives. In particular, the opiates included in the embodiments of the sustained-release opiate compositions may include adulmine, allocryptopine, aporphine, benzylmorphine, berberine, bicuculine, bicucine, bulbocapnine, buprenorphine, butorphanol, canadine, capaurine, chelerythrine, chelidonine, codamine, codeine, coptisine, coreximine, corlumine, corybulbine, corycavamine, corycavine, corydaline, corydine, corytuberine, cularine, cotamine, cryptopine, cycloartenol, cycloartenone, cyclolaudenol, dehydroreticuline, desomorphine, dextropropoxyphene, dextrorphanol, diacetylmorphine, dicentrine, dihydrosanguinarine, dipropanoylmorphine, epiporphyroxine, ethylmorphine, eupaverine, fagarine, fentanyl, glaucine, homochelidonoine, hydrocodone, hydrocotamine, hydromorphone, hydroxythebaine, isoboldine, isocorybulbine, isocorydine, isocorypalmine, isoquinoline, laudanidine, laudanine, laudanosine, levorphanol, magnoflorine, meconic acid, methadone, morphine, nalbuphine, nalmefene, naloxone, naltrexamine, α-naltrexol, β-naltrexol, naltrexone, naphthaphenanthridine, narceine, narceinone, narcotoline, narcotine, neopine, nicomorphine, norlaudanosoline, norsanguinarine, noscapine, opium, oripavine, oxycodone, oxymorphone, oxysanguinarine, palaudine, papaverine, papaveraldine, papaverrubine, perparin, pethidine, phenanthrene, phtalide-isoquinoline, porphyroxine, protopine, pseudocodeine, pseudomorphine, reticuline, salutaridine, sinoacutine, sanguinarine, scoulerine, somniferine, stepholidine, tapentadol, tetrahydroprotoberberine, thebaine, tramadol, and xanthaline. In exemplary embodiments, the opiate included in an embodiment of the sustained-release opiate compositions may be selected from oxycodone, oxymorphone, hydrocodone, hydromorphone, nalbuphine, naloxone, buprenorphine, and naltrexone.

Any of the opiates included in the embodiments of the sustained-release opiate compositions may have a (−) or (+) orientation with respect to the rotation of polarized light, depending upon whether the starting substrate has (−) or (+) optical activity. More specifically, each chiral center may independently have an R or an S configuration.

As an illustrative example, an embodiment of the sustained release opiate composition may include a morphinan compound. For the purposes of discussion, the ring atoms of a morphinan compound may be numbered as diagramed in Formula (I) below. Morphinan compounds have asymmetric centers and the core morphinan compound may have at least four chiral carbons: C-5, C-13, C-14, and C-9. In various embodiments, the configuration of the chiral carbons C-5, C-13, C-14, and C-9 may be RRRR, RRSR, RRRS, RRSS, RSRR, RSSR, RSRS, RSSS, SRRR, SRSR, SRRS, SRSS, SSRR, SSSR, SSRS, or SSSS, provided that the C-15 and the C-16 carbons are both either on the alpha face or the beta face of the molecule.

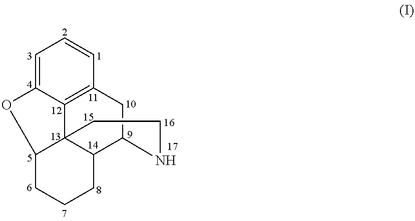

In various embodiments of the sustained-release opiate compositions, the opiate is attached to a connecting bridge that is also attached to a blood albumin binder. In one embodiment, the opiate is covalently bonded to the connecting bridge. The covalent bond used to attach the opiate to the connecting bridge is selected to allow the bond to be broken or cleaved after a specified time in the bloodstream, causing the opiate to be released in an active form into the bloodstream. The covalent bond used to attach the opiate to the connecting bridge is further selected such that the efficacy of the opiate in its active form is not significantly degraded due to chemical reactions such as hydrolysis used to break the covalent bond attaching the opiate to the connecting bridge. In an exemplary embodiment, the covalent bond used to attach the opiate to the connecting bridge is an ester bond that is hydrolyzed to release the opiate into the bloodstream in its active form.

The covalent bonds selected to attach the opiate to the connecting bridge in various embodiments are described in detail in Section III below.

### (II) Blood Albumin Binder

The blood albumin binders used in various embodiments of the sustained-release opiate composition include various non-peptide compounds having a strong affinity for binding to blood albumin. Without being bound to any particular theory, the blood albumin binder non-covalently binds to the blood albumin and effectively binds any other attached components of the sustained-release opiate composition to the blood albumin as well, including the opiate and the binding bridge.

In addition, blood albumin binders used in the sustained-release opiate compositions possess a specific affinity for blood albumin relative to other blood proteins or other tissue proteins, so as to avoid the binding of the sustained-release opiate composition to undesired proteins such as blood cell receptor proteins, chemokines, antibodies, or other circulating or non-circulating protein structures.

Blood albumin binders suitable for use in the sustained-release opiate compositions may include but are not limited to one or more functional groups. Non-limiting examples of suitable functional groups include aliphatic and aryl groups, in which each aliphatic and aryl group includes between about 1 and about 60 carbons, as well as any one or more substituents including but not limited to a nitrogen, an oxygen, a sulfur, a halogen, an alkyl group, an amide, an ester, and a sulfonamide. In an exemplary embodiment, the blood albumin binders may include but are not limited to diphenylcyclohexanol, phenylhexanol, biphenylpropanol, and 1,4-tetrahydronaphthalene.

The blood albumin binders included in the sustained-release opiate composition are covalently bonded to the connecting bridge in a manner that does not interfere with the release of the opiate into the bloodstream upon cleaving the connecting bridge. Covalent bonds suitable for attaching the blood albumin binder to the connecting bridge are described in detail in Section III below.

### (III) Connecting Bridge

The connecting bridge is attached to both the opiate and to the blood albumin binding bridge using covalent chemical bonds. The particular connecting bridges used in various embodiments of the sustained-release opiate composition are selected based on at least several criteria.

A connecting bridge may be selected so that it does not significantly interfere with the binding of the blood albumin binder with the blood albumin protein after administration of the sustained-release opiate composition. Interference with the binding of the blood albumin binder may be due to at least several factors including the direct influence of the connecting bridge on the chemical properties of the blood albumin binder, affinity of the binding bridge itself for blood albumin, and the interference of the opiate attached to the binding bridge with the blood albumin binder. The chemical properties of the connecting bridge such as electronegativity and the physical properties of the connecting bridge such as molecule length and bond flexibility are selected to minimize interference with the binding of the blood albumin binder to the circulating albumin protein.

In addition, the binding bridge is selected on the basis of its ability to degrade within an elapsed time in the bloodstream after administration of the sustained-release opiate composition. Because active opiate is released into the bloodstream after the cleaving of the bonds attaching the opiate to the binding bridge, this elapsed time determines the release profile of the opiate into the bloodstream. This desired elapsed time may range from a relatively short period of about one hour up to the half-life of blood albumin in the serum of a human patient, or about twenty days. In other embodiments, the binding bridge may be selected to degrade and release opiate into the bloodstream within a elapsed time ranging between about one hour and about four hours, between about two hours and about six hours, between about four hours and about twelve hours, between about six hours and about eighteen hours, between about twelve hours and about one day, between about eighteen hours and about three days, between about 2 days and about 4 days, between about 3 days and about 5 days, between about 4 days and about 8 days, between about 6 days and about 10 days, between about 8 days and about 12 days, between about 10 days and about 14 days, between about 12 days and about 16 days, and between about 15 days and about 20 days.

Further, the binding bridge is selected such that the cleaving of the bond attaching the binding bridge to the opiate does not degrade the efficacy of the active opiate in the bloodstream relative to the efficacy of the opiate administered in isolation.

Suitable compounds for use as binding bridges in various embodiments include but are not limited to functional groups chosen from phosphate monoesters, phosphate diesters, phosphate triesters, carbonates, sulphonates, boric acid esters, and diesters of dicarboxylic acids such as oxalic, citric, glutaric, tartaric, malonic, aspartic, glutamic, suberic, fumaric, maleic, succinic, and adipic acids. In an exemplary embodiment, the binding bridge includes phosphate diesters as a functional group.

In an embodiment, the binding bridge is attached to the opiate using an ester bond formed at a hydroxy group attached to the opiate. The hydroxy group attached to the opiate may be a group that naturally occurs on the opiate, or the hydroxy group may be added to the opiate for the purpose of attaching the binding bridge. The place of attachment of the binding bridge to the opiate may affect the degradation of the bond connecting the binding bridge to the opiate due to the chemical interaction of various moieties of the opiate that are in close proximity to the place of attachment. In another embodiment, the place of attachment of the connecting bridge to the opiate may be selected in order to achieve a desired pharmacokinetic profile.

As an illustrative example, if the composition includes oxymorphone, shown as Formula (II) below, the binding bridge may be attached at the hydroxy group attached at C-3 or at C-14. In addition, the binding bridge may be attached at the oxygen attached at C-6.

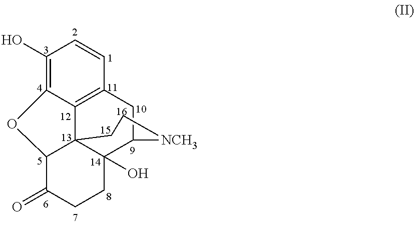

In another illustrative example, if the composition includes buprenorphine, shown as Formula (IIA) below, the binding bridge may be attached at the hydroxy group attached at C-3 or at C-20.

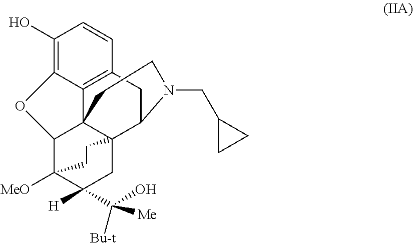

In other embodiments, the sustained-release opiate composition may include compounds made up of more than one binding bridge functional group in order to specify the desired opiate pharmacokinetic release profile. For example, a sustained-release opiate composition may include a mixture of compounds that include binding bridges that degrade after a relatively short elapsed time as well as compounds that include binding bridges that degrade after a relatively longer elapsed time. Upon administration, the opiates attached to the shorter-lived connecting bridges would be released shortly after administration. As the active opiate is metabolized and otherwise removed from circulation, additional opiate attached to the longer-lived connecting bridges would be released. In this embodiment, a more sustained and uniform opiate pharmacokinetic release profile results from the replacement of the metabolized opiate with newly released opiate.

### (IV) Methods of Making Sustained Release Opiate Compositions

The sustained-release opiate composition may be made using any techniques known in the art. As an illustrative example, the sustained-release opiate composition may be made using techniques disclosed by U.S. Pat. No. 6,676,929, which is hereby incorporated by reference herein in its entirety. Although the specific steps of the synthesis process may vary depending on at least several factors including the specific opiate compound, blood albumin binder, and connecting bridge, the process disclosed by U.S. Pat. No. 6,676,929 follows the general scheme illustrated below for a phosphate diester binding bridge:

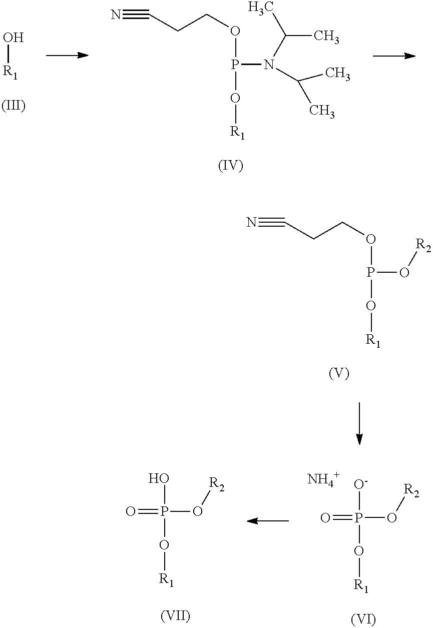

in which:
R₁ is an opiate, described in Section (I) above, to be included in the sustained-release opiate composition, and
R₂ is a blood albumin binder, described in Section (II) above, to be included in the sustained-release opiate composition.

In general, the opiate R1 is combined with 2-cyanoethyl N,N-diisopropylchlorophosphoramidite, resulting in a phosphoramidite intermediate product, shown as Formula (IV) above. The amidite moiety is replaced by the blood albumin-binding moiety to yield a phosphate intermediate product, shown as Formula (V) above, which still includes an attached cyanoethyl group. The cyanoethyl group is subsequently replaced by an ionically bonded ammonium ion, as shown in Formula (VI) above. A strong acid is added to displace the ammonium ion with a hydrogen anion, resulting in the sustained-release opiate composition, shown above in Formula (VII).

Although the method of making the sustained release opiate composition illustrated above included a phosphate diester as the binding bridge, any one of the binding bridges described in Section (III) above may be used in other embodiments of the method with appropriate modifications.

For the synthesis of sustained-release opiate compounds that include opiates possessing more than one attached hydroxy group, those hydroxy groups for which no attachment of blood albumin binders is desired may be chemically modified with moieties such as t-butyl groups to prevent the attachment of blood albumin binders during the synthesis of the sustained-release opiate compositions. Yet other embodiments may further include the attachment of a hydroxy group on the opiate compound at a specified position to act as a point of attachment for the blood albumin binder.

As an illustrative example, a method of making a sustained-release oxymorphone composition by attaching the oxymorphone to a diphenylcyclohexanol blood albumin binder using a phosphate diester connecting bridge is described as follows. 2-cyanoethyl N,N-diisopropylchlorophosphoramidite is added to a stirred solution of oxymorphone and diisopropylethylamine in distilled CH₂Cl₂ at room temperature, and the resulting solution is stirred at room temperature for 2 hours. After stirring, the solution is diluted with CH₂Cl₂ and then washed with ice-cold 10% NaHCO₃ solution, H₂O, and brine, and dried over MgSO₄. The organic layer of the solution is then removed and evaporated to obtain a crude phosphoramidite intermediate product, shown as Formula (VIII), in the form of a pale yellow oil:

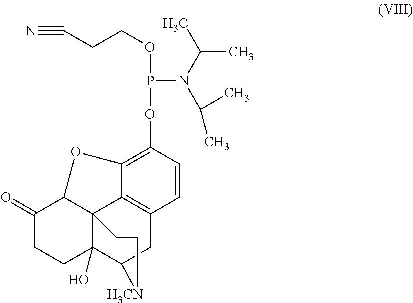

The phosphoramidite intermediate in distilled CH₃CN is combined with 10-phenyl-1-decanol and 1H-tetrazole in distilled CH₃CN and then reacted with T-butylhydroperoxide for 1 hour at room temperature. The solvent is then concentrated in vacuo and the residue is portioned between AcOEt and H₂O. The organic layer is washed with H₂O and NaCl (sat.), dried over MgSO₄ and evaporated. The residue is then purified with silica gel column chromatography to give the 4,4-diphenylcyclohexyloxy phosphate product shown as Formula (IX):

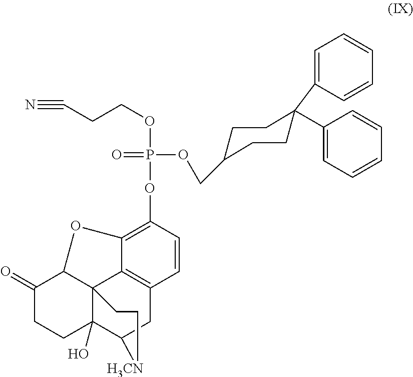

The 4,4-diphenylcyclohexyloxy phosphate product was combined with 2M NH₃—MeOH and stirred at room temperature for 5 hours. The solvent was evaporated, leaving a 4,4-diphenylcyclohexyl phosphodiester product, shown below as Formula (X):

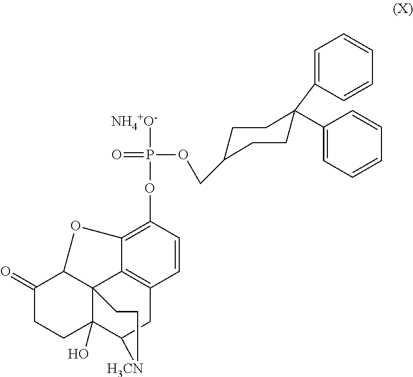

The 4,4-diphenylcyclohexyl phosphodiester product is mixed with cHCl (trace metal grade) and ether and stirred at room temperature overnight. The solvents are evaporated off and the residue is triturated with H₂O. The resulting precipitate was filtered and washed with H₂O and ether. The solid product was dried under vacuum at room temperature for 24 hours to give the pure sustained-release oxymorphone product, shown as Formula (XI):

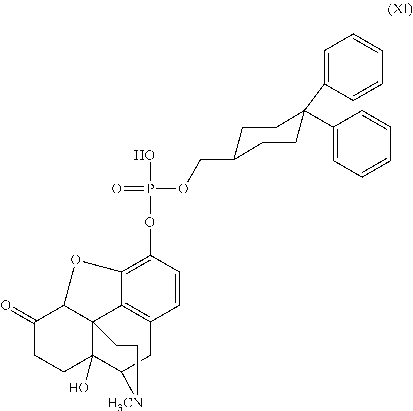

### (IV) Methods of Administration

An embodiment of the present invention provides a method of administering an opiate to a human subject with a sustained release profile relative to a pharmacokinetic profile of the opiate administered in isolation. The method includes providing a sustained-release opiate composition that includes an opiate, a non-peptide blood albumin binder, and a connecting bridge attached to the opiate and to the non-peptide blood albumin binder such as the compositions described above. The method further includes administering the sustained-release opiate composition to the human subject. The sustained-release opiate composition may be administered by methods including but not limited to intravenous injection, intramuscular injection, infusion, transdermal absorption, ingestion, inhalation, vaginal absorption and rectal absorption. In an exemplary embodiment, the sustained-release opiate composition is administered by intravenous injection.

Because the opiate is released with a sustained-release pharmacokinetic profile, the sustained-release opium composition may be administered to the human subject at an effective dosage that is higher than the recommended dosage for the corresponding opiate administered in isolation. Upon administration, at least some fraction of the opiate included in the sustained-release opiate composition is bound to the blood albumin of the human subject in an inactive form for later release. Therefore, in order to achieve a therapeutically effective blood concentration of opiate, a higher dosage may be administered. However, because the opiate is released in active form with a sustained release pharmacokinetic profile, the sustained-release opiate composition may be administered less frequently compared to the corresponding opiate administered in isolation.

Formulations of the various embodiments of the sustained-release opiate compositions are dependent on a number of factors including but not limited to the method of administration of the composition. As an illustrative example, a formulation of the sustained-release opiate composition to be administered by ingestion may include excipients such as binders and taste masking agents. Formulations of various embodiments of the sustained-release opiate composition are described below.

### (V) Formulations

Formulations of various embodiments may include the sustained-release opiate composition, along with an excipient. Non-limiting examples of excipients include binders, fillers, non-effervescent disintegrants, effervescent disintegration agents, preservatives, diluents, flavor-modifying agents, sweeteners, lubricants, dispersants, coloring agents, taste masking agents, pH modifiers. and combinations of any of these agents.

Non-limiting examples of binders suitable for the formulations of various embodiments include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohols, polyethylene glycol, polyols, saccharides, oligosaccharides, polypeptides, oligopeptides, and combinations thereof. The polypeptide may be any arrangement of amino acids ranging from about 100 to about 300,000 Daltons.

Non-limiting examples of fillers include carbohydrates, inorganic compounds, and polyvinylpirrolydone. Other non-limiting examples of fillers include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, lactose, sucrose, mannitol, and sorbitol.

Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

Non-limiting examples of preservatives include antioxidants, such as a-tocopherol or ascorbate, and antimicrobials, such as parabens, chlorobutanol or phenol.

Diluents suitable for use include but are not limited to pharmaceutically acceptable saccharides such as sucrose, dextrose, lactose, microcrystalline cellulose, fructose, xylitol, and sorbitol; polyhydric alcohols; starches; pre-manufactured direct compression diluents; and mixtures of any of the foregoing.

Suitable flavor-modifying agents include but are not limited to synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevie rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Non-limiting examples of lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil.

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in various embodiments.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking agents contemplated are those described in U.S. Pat. Nos. 4,851,226, 5,075,114, and 5,876,759, each of which is hereby incorporated by reference in its entirety.

Non-limiting examples of pH modifiers include sodium carbonate and sodium bicarbonate.

In those embodiments administered orally, the sustained-release opiate compositions may be administered in any orally acceptable dosage form including, but not limited to, capsules, tablets, aqueous suspensions, or solutions. Capsule and tablet formulations may include, but are not limited to binders, lubricants, and diluents. Aqueous suspension formulations may include but are not limited to dispersants, flavor-modifying agents, taste-masking agents, and coloring agents.

For those embodiments using rectal absorption as a method of administration, the sustained-release opiate composition may be administered in the form of rectal suppositories. In these embodiments, the composition may include a suitable non-irritating excipient that is solid at room temperature but liquid at rectal temperature and therefore will melt in the rectum to release the drug. Non-limiting examples of suitable excipients for rectal suppository embodiments include cocoa butter, beeswax, and polyethylene glycols.

For transdermally absorbed embodiments, the composition may be formulated as a suitable ointment, lotion, or cream that includes but is not limited to the sustained-release opiate composition suspended or dissolved in one or more carriers. Non-limiting examples of suitable carriers for transdermal embodiments include mineral oil, liquid petrolatum, white petrolatum, propylene glycol, polyoxyethylene, polyoxypropylene compound, emulsifying wax, sorbitan monostearate, Polysorbate 60, cetyl esters wax, cetearyl alcohol, 2-octyldodecanol, benzyl alcohol and water. For these embodiments, the molecular weight of the composition may range from about 1 to about 50 Daltons.

### (V) Exemplary Embodiments

The molecules of various embodiments of the sustained-release opiate compositions are described generally by Formula (XII) below:

A-B-X (XII);

in which A is a blood albumin binder described in Section II, B is a connecting bridge described in Section III, and X is an opiate described in Section I. For those opiates having more than one hydroxy group, the connecting bridge B may attach to the opiate X at any one of its hydroxy groups, as described in Section (III) above. In other embodiments, a hydroxy group may be attached to the opiate X at a specified location to provide a binding site for the binding bridge B. Because the moieties in the vicinity of the point of attachment of the binding bridge B to the opiate X may affect the release properties of the opiate upon administration, a composition in which the connecting bridge is connected at one hydroxy group on the opiate is considered a different embodiment compared to a composition in which the connecting bridge is connected to a different hydroxy group at a different location on the opiate.

Non-limiting examples of embodiments of the sustained-release opiate compositions are listed in Table I below:

TABLE I
Exemplary Embodiments of Sustained-Release Opiate Compositions

| Blood Albumin | Connecting | | |
|---|---|---|---|
| Binder (A) | Bridge (B) | Opiate (X) | Chemical Structure |
| diphenylcyclohexanol | phosphate diester attached at C-3 | oxymorphone | 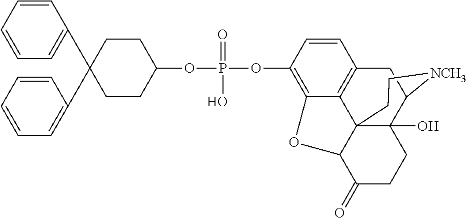 |
| diphenylcyclohexanol | phosphate diester attached at C-14 | oxymorphone | 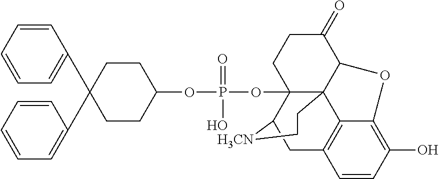 |
| phenylhexanol | phosphate diester attached at C-3 | oxymorphone | 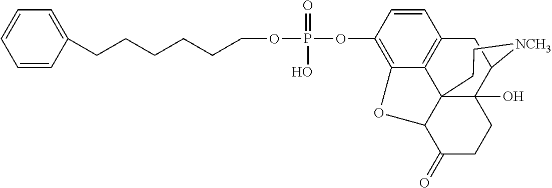 |
| phenylhexanol | phosphate diester attached at C-14 | oxymorphone | 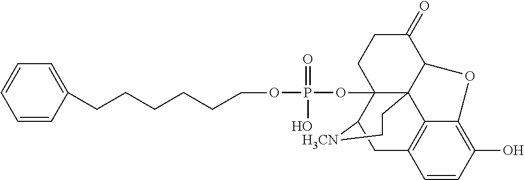 |
| biphenylpropanol | phosphate diester attached at C-3 | oxymorphone | 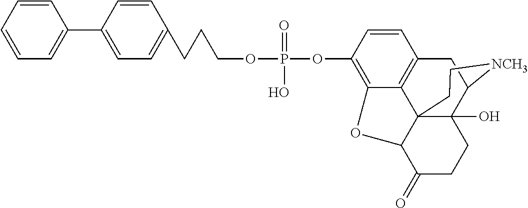 |
| biphenylpropanol | phosphate diester attached at C-14 | oxymorphone | 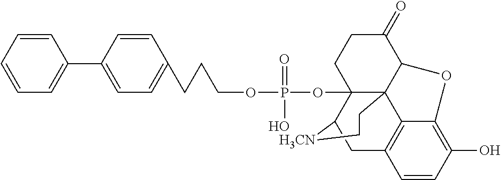 |
| 1,4- tetrahydronaphthalene | phosphate diester attached at C-3 | oxymorphone | 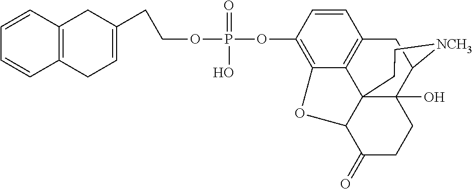 |
| 1,4- tetrahydronaphthalene | phosphate diester attached at C-14 | oxymorphone | 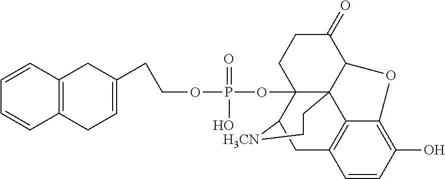 |
| diphenylcyclohexanol | carbonate attached at C-3 | oxymorphone | 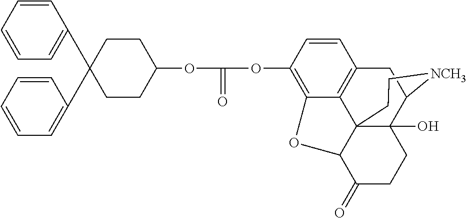 |
| diphenylcyclohexanol | carbonate attached at C-14 | oxymorphone | 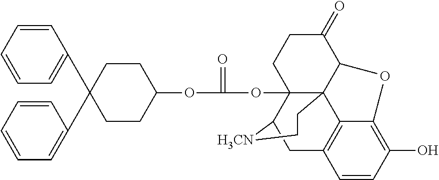 |
| phenylhexanol | carbonate attached at C-3 | oxymorphone | 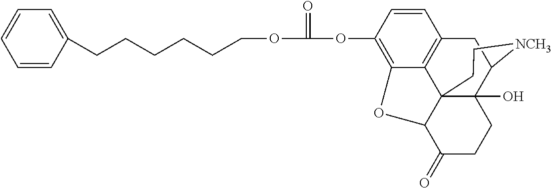 |
| phenylhexanol | carbonate attached at C-14 | oxymorphone | 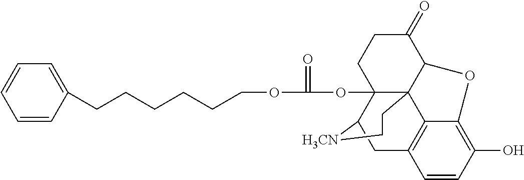 |
| biphenylpropanol | carbonate attached at C-3 | oxymorphone | 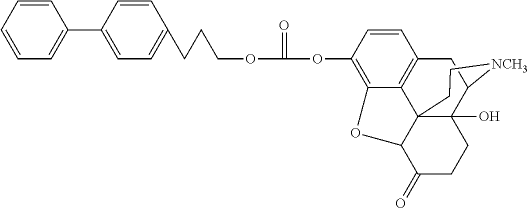 |
| biphenylpropanol | carbonate attached at C-14 | oxymorphone | 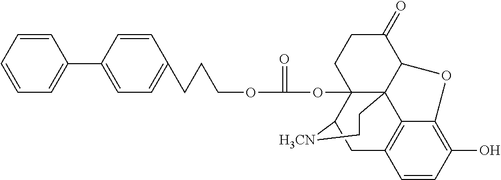 |
| 1,4- tetrahydronaphthalene | carbonate attached at C-3 | oxymorphone | 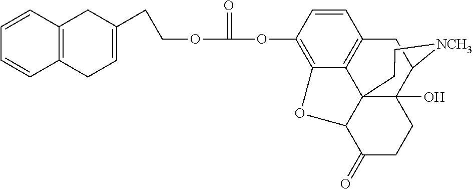 |
| 1,4- tetrahydronaphthalene | carbonate attached at C-14 | oxymorphone | 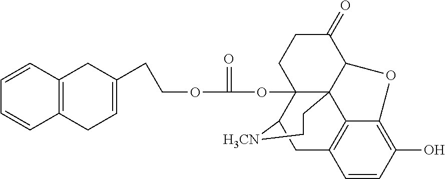 |
| diphenylcyclohexanol | sulphonate attached at C-3 | oxymorphone | 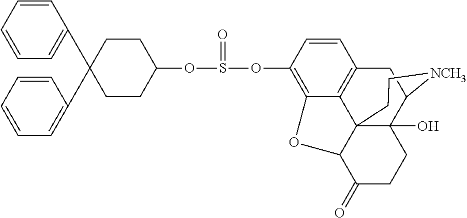 |
| diphenylcyclohexanol | sulphonate attached at C-14 | oxymorphone | 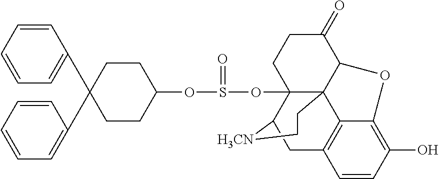 |
| phenylhexanol | sulphonate attached at C-3 | oxymorphone | 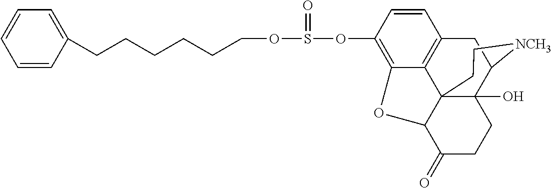 |
| phenylhexanol | sulphonate attached at C-14 | oxymorphone | 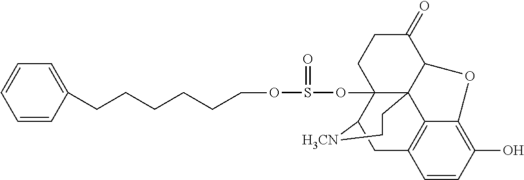 |
| biphenylpropanol | sulphonate attached at C-3 | oxymorphone | 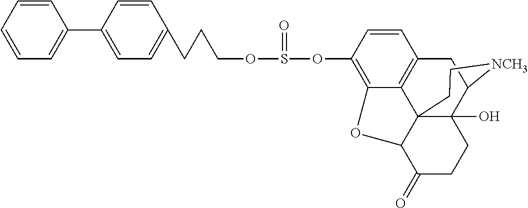 |
| biphenylpropanol | sulphonate attached at C-14 | oxymorphone | 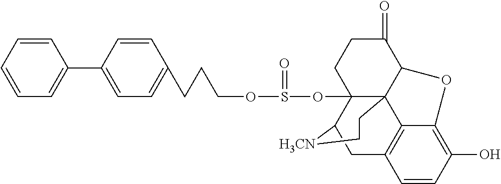 |
| 1,4- tetrahydronaphthalene | sulphonate attached at C-3 | oxymorphone | 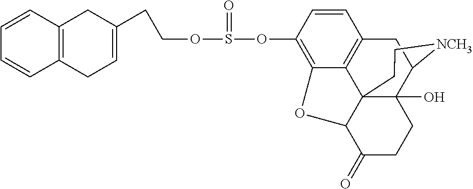 |
| 1,4- tetrahydronaphthalene | sulphonate attached at C-14 | oxymorphone | 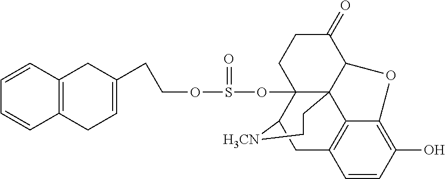 |
| diphenylcyclohexanol | phosphate diester | oxycodone | 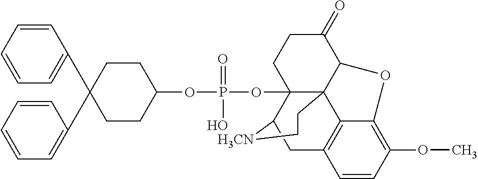 |
| phenylhexanol | phosphate diester | oxycodone | 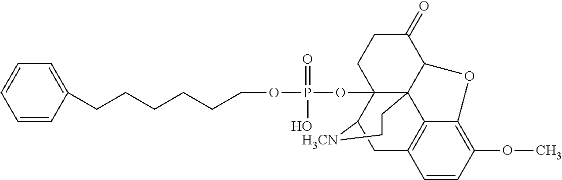 |
| biphenylpropanol | phosphate diester | oxycodone | 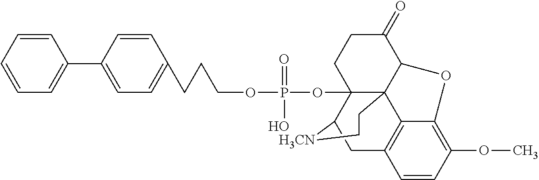 |
| 1,4- tetrahydronaphthalene | phosphate diester | oxycodone | 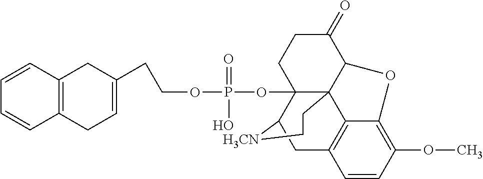 |
| diphenylcyclohexanol | carbonate | oxycodone | 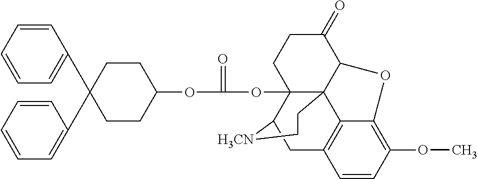 |
| phenylhexanol | carbonate | oxycodone | 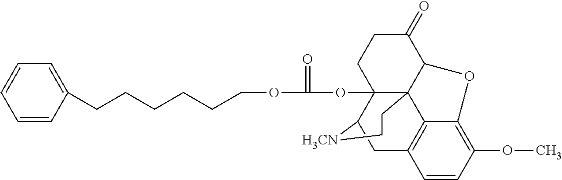 |
| biphenylpropanol | carbonate | oxycodone | 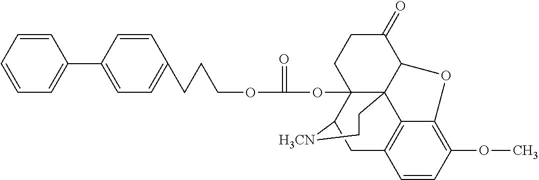 |
| 1,4- tetrahydronaphthalene | carbonate | oxycodone | 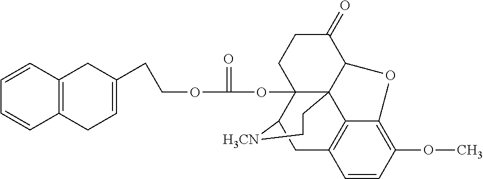 |
| diphenylcyclohexanol | sulphonate | oxycodone | 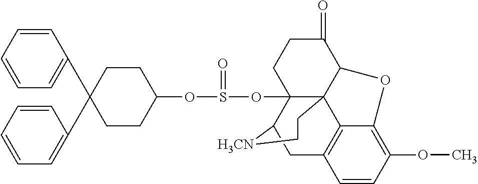 |
| phenylhexanol | sulphonate | oxycodone | 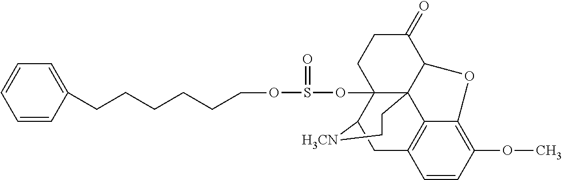 |
| biphenylpropanol | sulphonate | oxycodone | 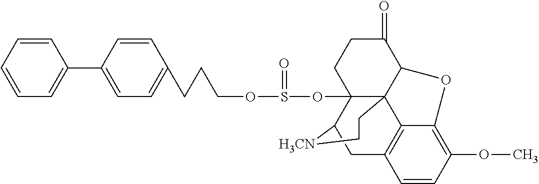 |
| 1,4- tetrahydronaphthalene | sulphonate | oxycodone | 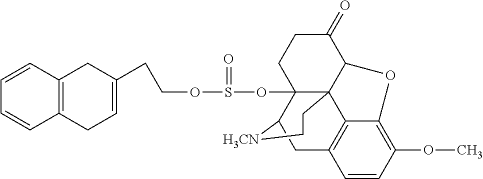 |
| diphenylcyclohexanol | phosphate diester | codeine | 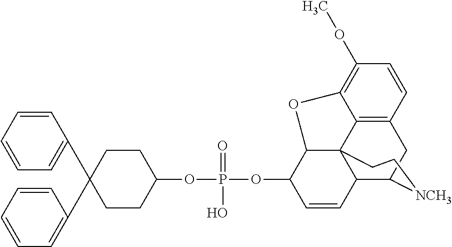 |
| phenylhexanol | phosphate diester | codeine | 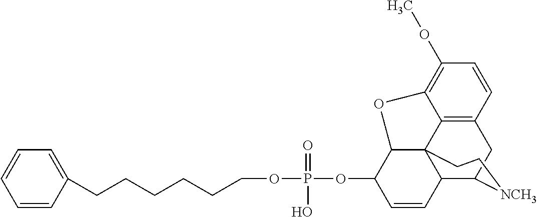 |
| biphenylpropanol | phosphate diester | codeine | 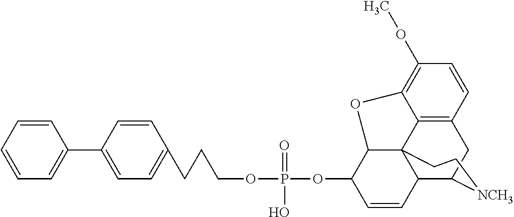 |
| 1,4- tetrahydronaphthalene | phosphate diester | codeine | 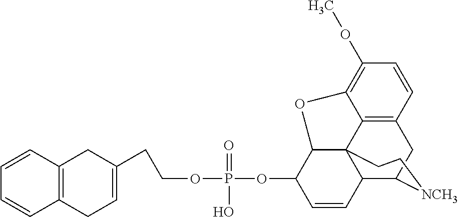 |
| diphenylcyclohexanol | carbonate | codeine | 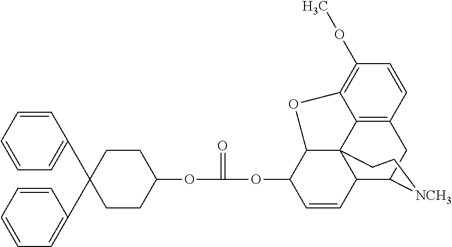 |
| phenylhexanol | carbonate | codeine | 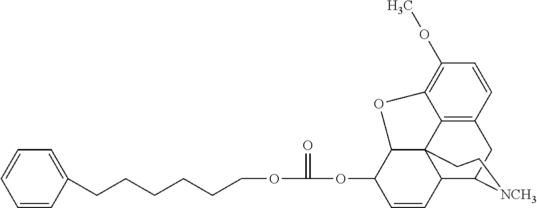 |
| biphenylpropanol | carbonate | codeine | 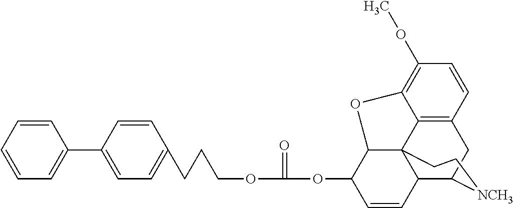 |
| 1,4- tetrahydronaphthalene | carbonate | codeine | 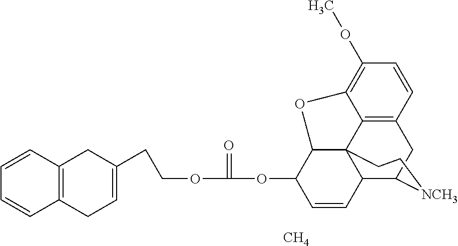 |
| diphenylcyclohexanol | sulphonate | codeine | 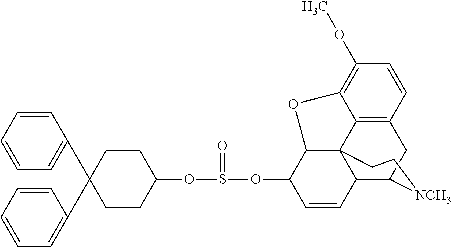 |
| phenylhexanol | sulphonate | codeine | 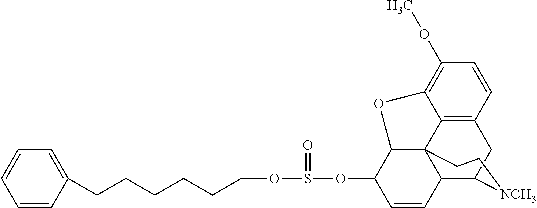 |
| biphenylpropanol | sulphonate | codeine | 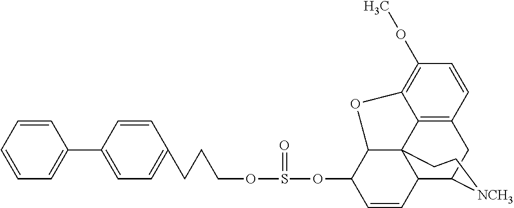 |
| 1,4- tetrahydronaphthalene | sulphonate | codeine | 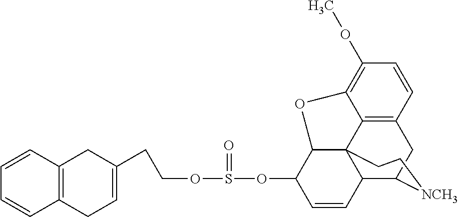 |
| diphenylcyclohexanol | phosphate diester attached at C-3 | naltrexone | 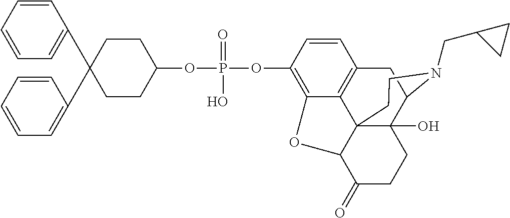 |
| diphenylcyclohexanol | phosphate diester attached at C-14 | naltrexone | 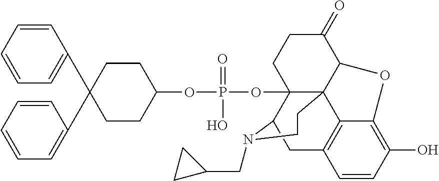 |
| phenylhexanol | phosphate diester attached at C-3 | naltrexone | 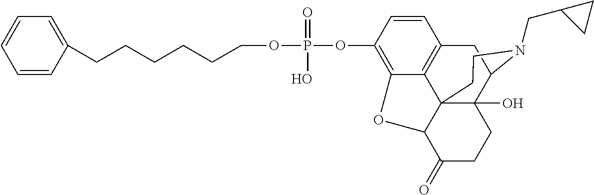 |
| phenylhexanol | phosphate diester attached at C-14 | naltrexone | 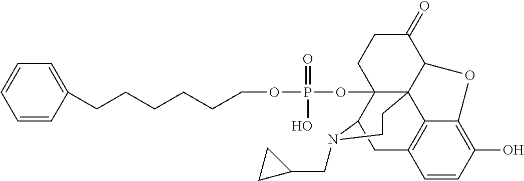 |
| biphenylpropanol | phosphate diester attached at C-3 | naltrexone | 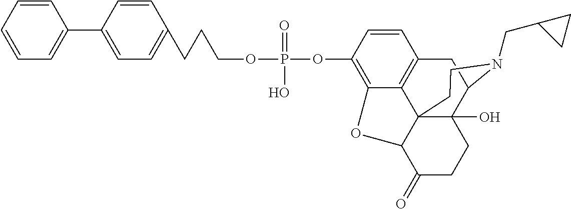 |
| biphenylpropanol | phosphate diester attached at C-14 | naltrexone | 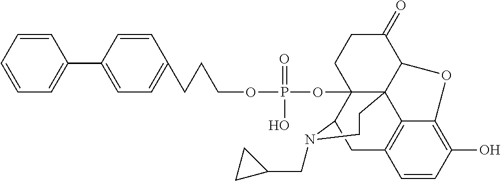 |
| 1,4- tetrahydronaphthalene | phosphate diester attached at C-3 | naltrexone | 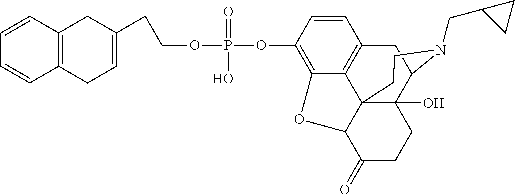 |
| 1,4- tetrahydronaphthalene | phosphate diester attached at C-14 | naltrexone | 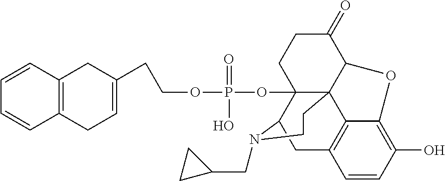 |
| diphenylcyclohexanol | carbonate attached at C-3 | naltrexone | 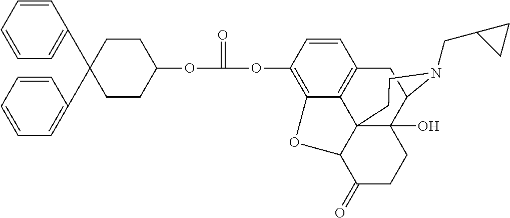 |
| diphenylcyclohexanol | carbonate attached at C-14 | naltrexone | 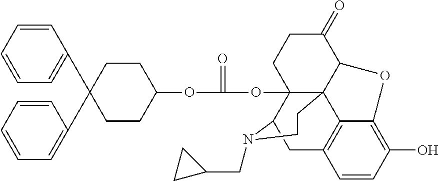 |
| phenylhexanol | carbonate attached at C-3 | naltrexone | 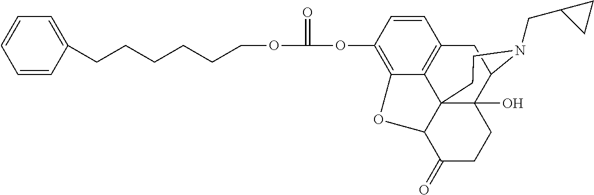 |
| phenylhexanol | carbonate attached at C-14 | naltrexone | 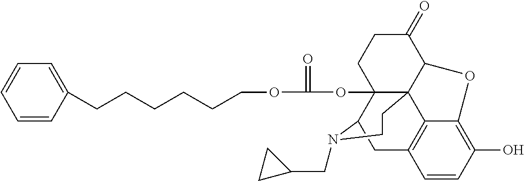 |
| biphenylpropanol | carbonate attached at C-3 | naltrexone | 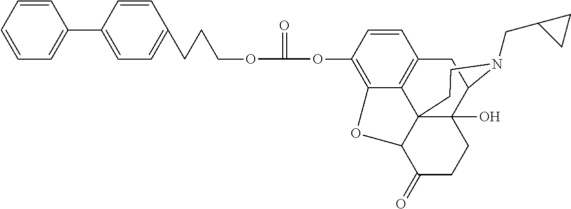 |
| biphenylpropanol | carbonate attached at C-14 | naltrexone | 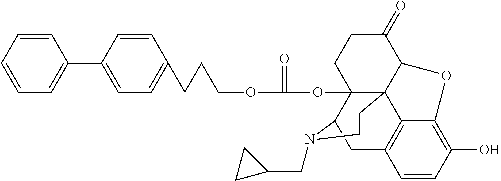 |
| 1,4- tetrahydronaphthalene | carbonate attached at C-3 | naltrexone | 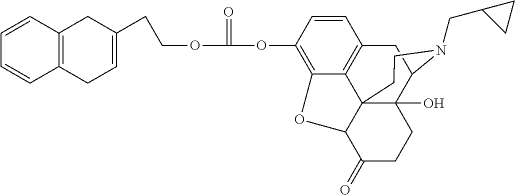 |
| 1,4- tetrahydronaphthalene | carbonate attached at C-14 | naltrexone | 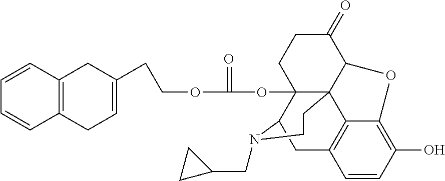 |
| diphenylcyclohexanol | sulphonate attached at C-3 | naltrexone | 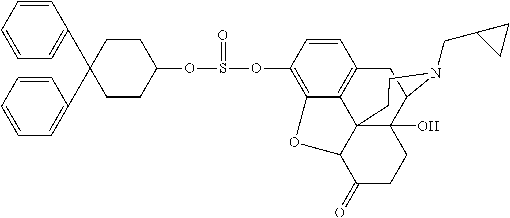 |
| diphenylcyclohexanol | sulphonate attached at C-14 | naltrexone | 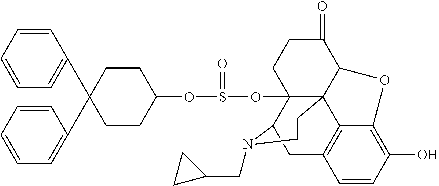 |
| phenylhexanol | sulphonate attached at C-3 | naltrexone | 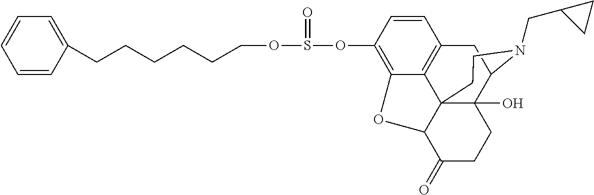 |
| phenylhexanol | sulphonate attached at C-14 | naltrexone | 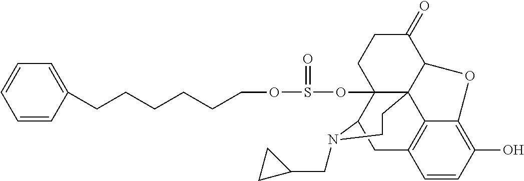 |
| biphenylpropanol | sulphonate attached at C-3 | naltrexone | 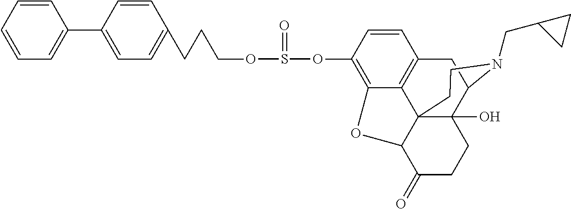 |
| biphenylpropanol | sulphonate attached at C-14 | naltrexone | 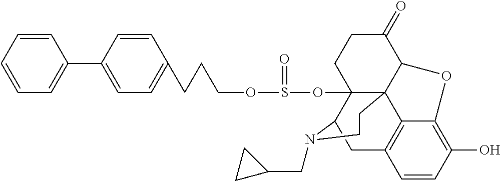 |
| 1,4- tetrahydronaphthalene | sulphonate attached at C-3 | naltrexone | 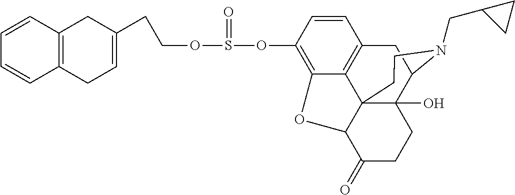 |
| 1,4- tetrahydronaphthalene | sulphonate attached at C-14 | naltrexone | 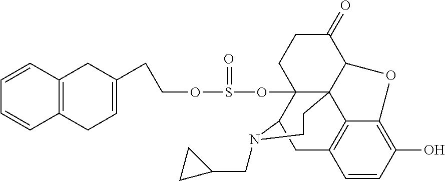 |
| diphenylcyclohexanol | phosphate diester attached at C-3 | morphine | 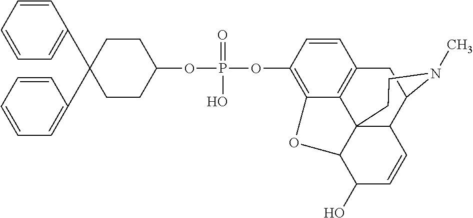 |
| diphenylcyclohexanol | phosphate diester attached at C-6 | morphine | 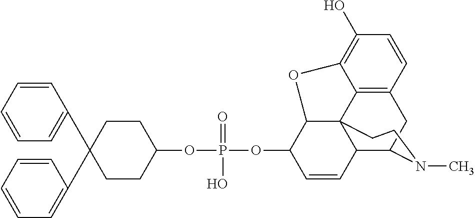 |
| phenylhexanol | phosphate diester attached at C-3 | morphine | 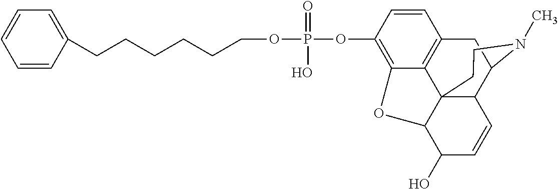 |
| phenylhexanol | phosphate diester attached at C-6 | morphine | 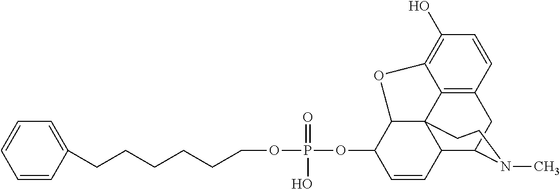 |
| biphenylpropanol | phosphate diester attached at C-3 | morphine | 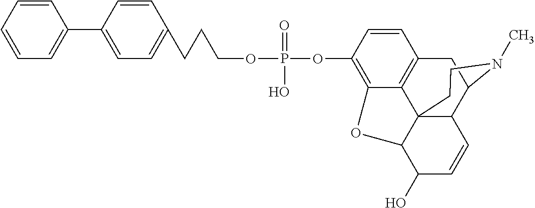 |
| biphenylpropanol | phosphate diester attached at C-6 | morphine | 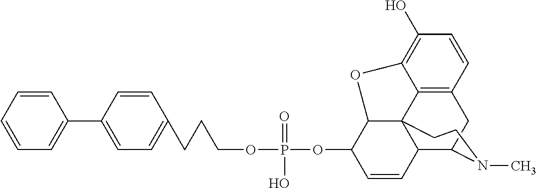 |
| 1,4- tetrahydronaphthalene | phosphate diester attached at C-3 | morphine | 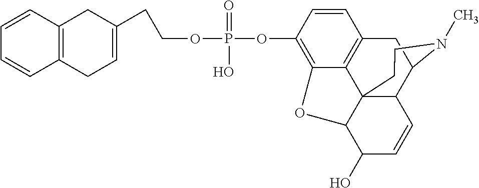 |
| 1,4- tetrahydronaphthalene | phosphate diester attached at C-6 | morphine | 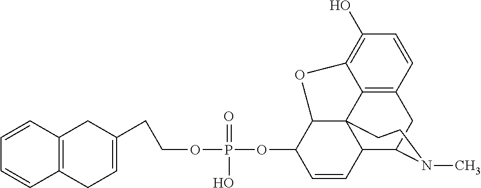 |
| diphenylcyclohexanol | carbonate attached at C-3 | morphine | 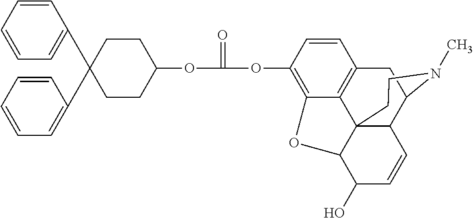 |
| diphenylcyclohexanol | carbonate attached at C-6 | morphine | 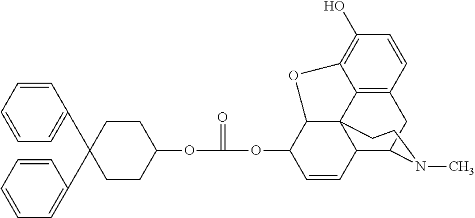 |
| phenylhexanol | carbonate attached at C-3 | morphine | 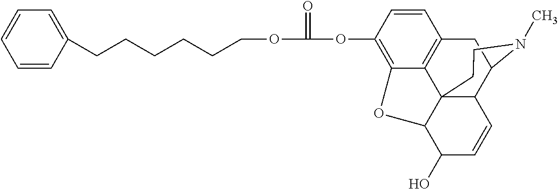 |
| phenylhexanol | carbonate attached at C-6 | morphine | 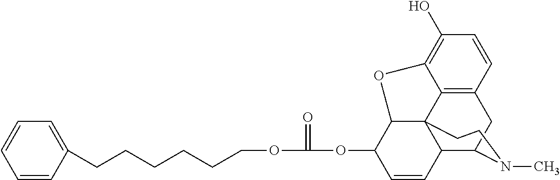 |
| biphenylpropanol | carbonate attached at C-3 | morphine | 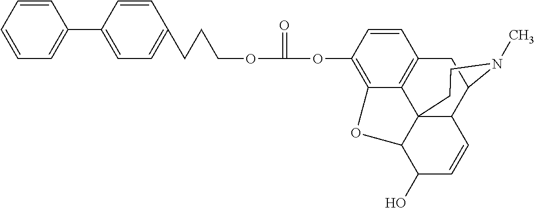 |
| biphenylpropanol | carbonate attached at C-6 | morphine | 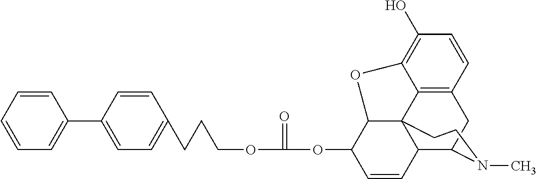 |
| 1,4- tetrahydronaphthalene | carbonate attached at C-3 | morphine | 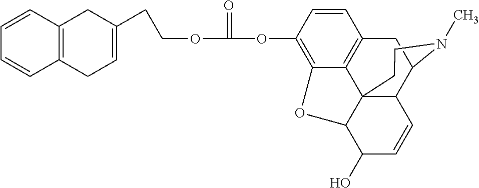 |
| 1,4- tetrahydronaphthalene | carbonate attached at C-6 | morphine | 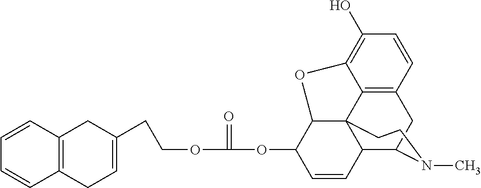 |
| diphenylcyclohexanol | sulphonate attached at C-3 | morphine | 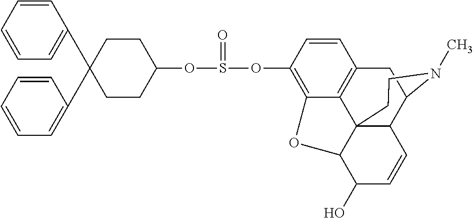 |
| diphenylcyclohexanol | sulphonate attached at C-6 | morphine | 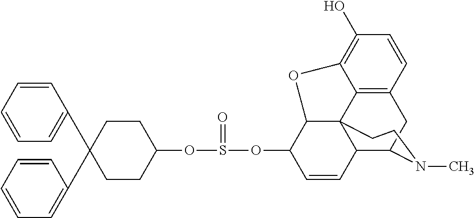 |
| phenylhexanol | sulphonate attached at C-3 | morphine | 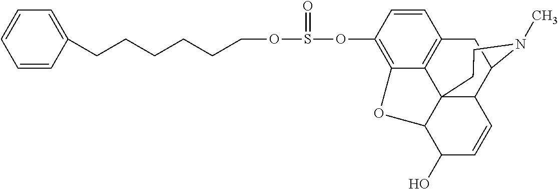 |
| phenylhexanol | sulphonate attached at C-6 | morphine | 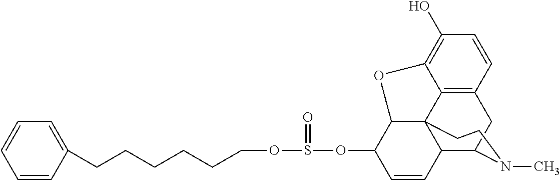 |
| biphenylpropanol | sulphonate attached at C-3 | morphine | 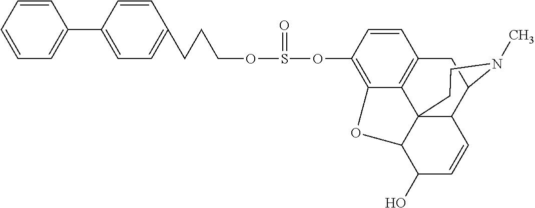 |
| biphenylpropanol | sulphonate attached at C-6 | morphine | 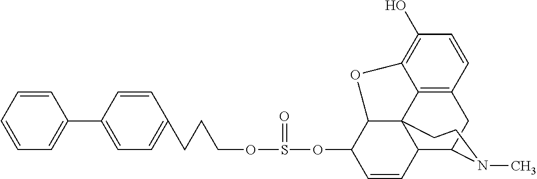 |
| 1,4- tetrahydronaphthalene | sulphonate attached at C-3 | morphine | 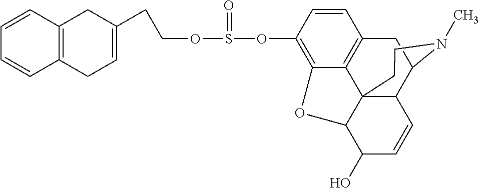 |
| 1,4- tetrahydronaphthalene | sulphonate attached at C-6 | morphine | 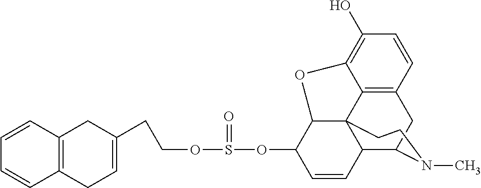 |
| diphenylcyclohexanol | phosphate diester attached at C-3 | buprenorphine | 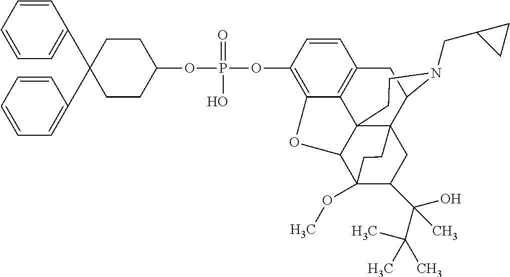 |
| diphenylcyclohexanol | phosphate diester attached at C-6 | buprenorphine | 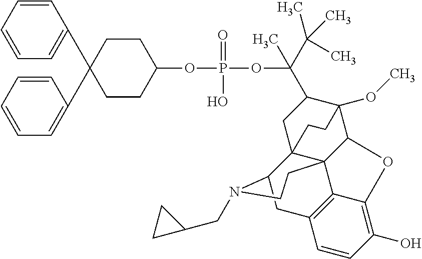 |
| phenylhexanol | phosphate diester attached at C-3 | buprenorphine | 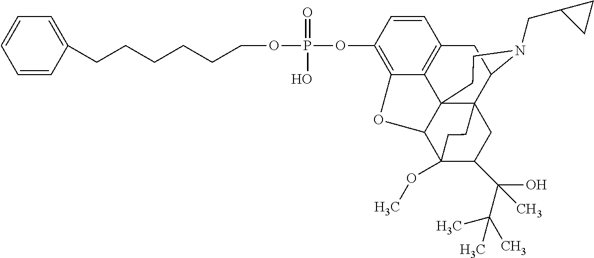 |
| phenylhexanol | phosphate diester attached at C-6 | buprenorphine | 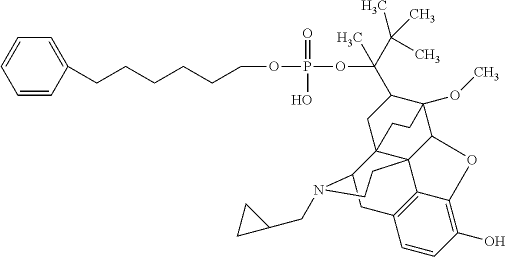 |
| biphenylpropanol | phosphate diester attached at C-3 | buprenorphine | 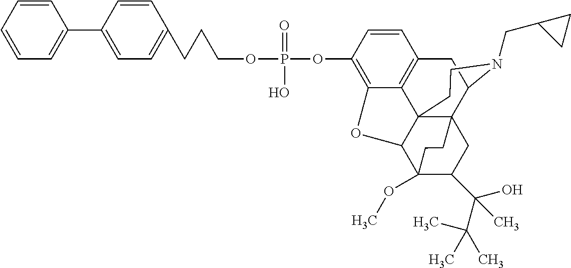 |
| biphenylpropanol | phosphate diester attached at C-6 | buprenorphine | 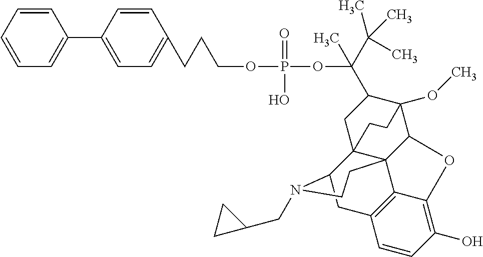 |
| 1,4- tetrahydronaphthalene | phosphate diester attached at C-3 | buprenorphine | 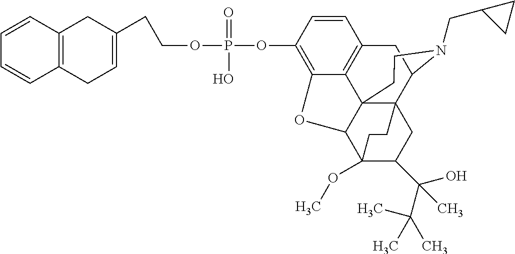 |
| 1,4- tetrahydronaphthalene | phosphate diester attached at C-6 | buprenorphine | 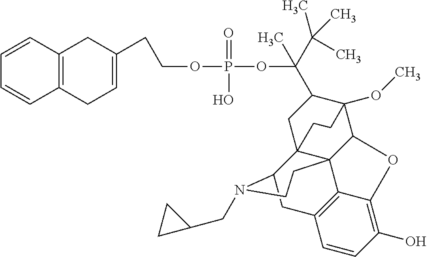 |
| diphenylcyclohexanol | carbonate attached at C-3 | buprenorphine | 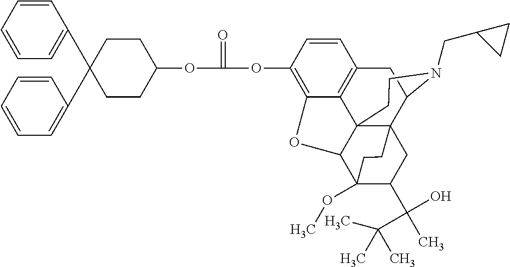 |
| diphenylcyclohexanol | carbonate attached at C-6 | buprenorphine | 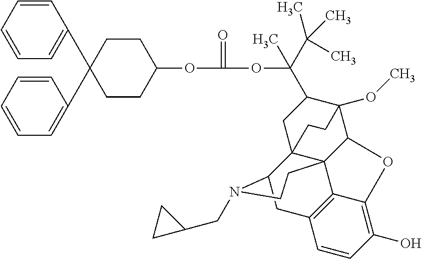 |
| phenylhexanol | carbonate attached at C-3 | buprenorphine | 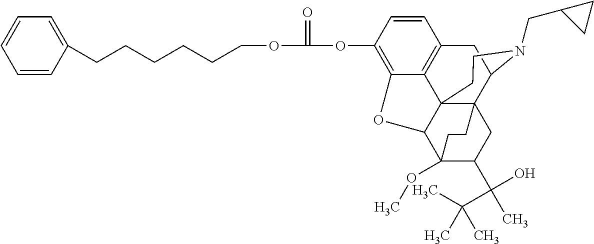 |
| phenylhexanol | carbonate attached at C-6 | buprenorphine | 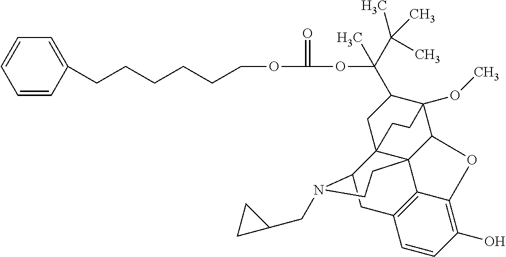 |
| biphenylpropanol | carbonate attached at C-3 | buprenorphine | 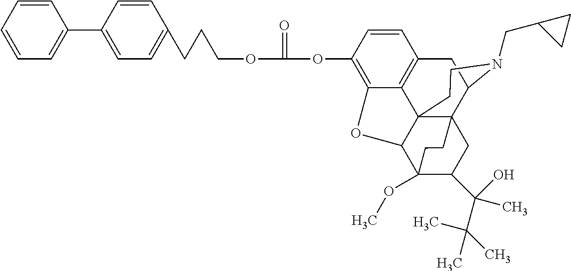 |
| biphenylpropanol | carbonate attached at C-6 | buprenorphine | 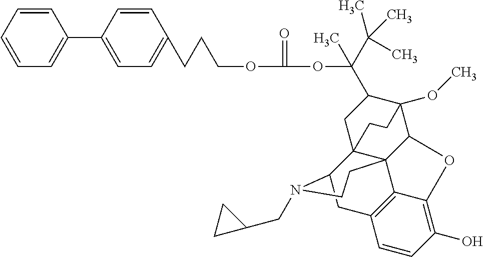 |
| 1,4- tetrahydronaphthalene | carbonate attached at C-3 | buprenorphine | 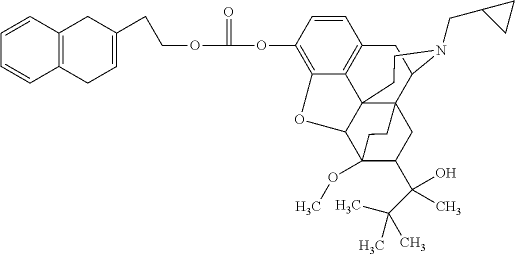 |
| 1,4- tetrahydronaphthalene | carbonate attached at C-6 | buprenorphine | 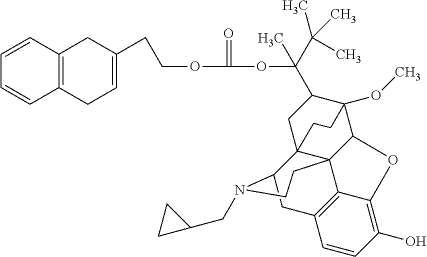 |
| diphenylcyclohexanol | sulphonate attached at C-3 | buprenorphine | 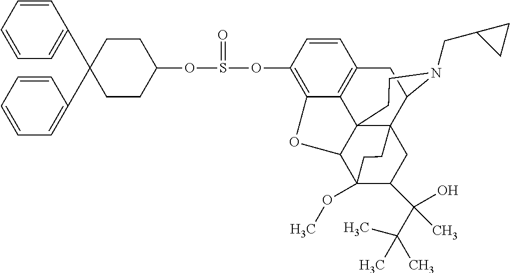 |
| diphenylcyclohexanol | sulphonate attached at C-6 | buprenorphine | 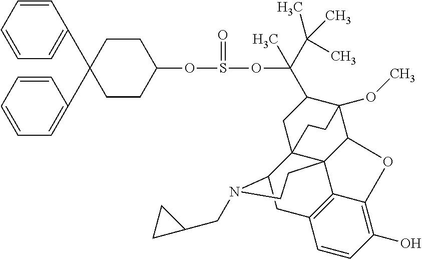 |
| phenylhexanol | sulphonate attached at C-3 | buprenorphine | 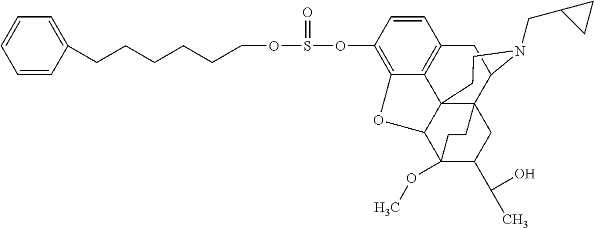 |
| phenylhexanol | sulphonate attached at C-6 | buprenorphine | 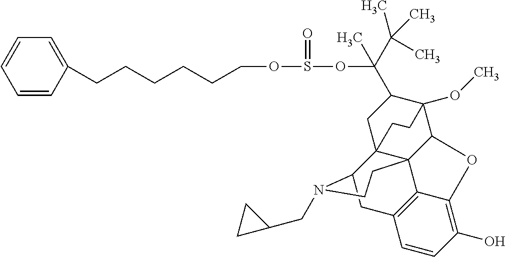 |
| biphenylpropanol | sulphonate attached at C-3 | buprenorphine | 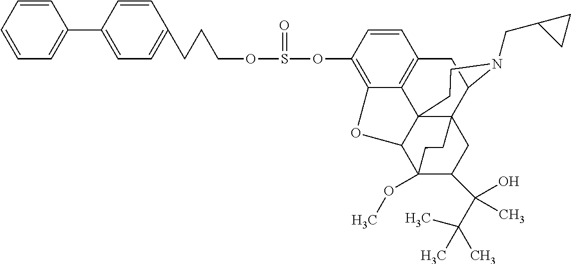 |
| biphenylpropanol | sulphonate attached at C-6 | buprenorphine | 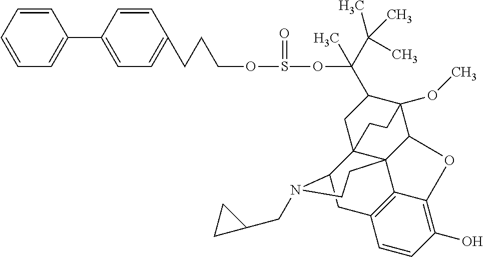 |
| 1,4- tetrahydronaphthalene | sulphonate attached at C-3 | buprenorphine | 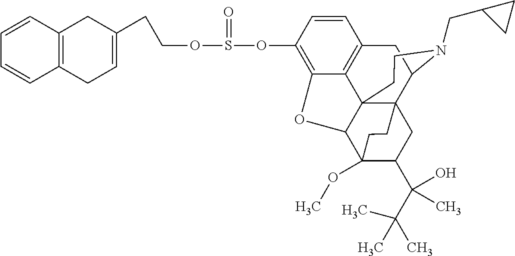 |
| 1,4- tetrahydronaphthalene | sulphonate attached at C-6 | buprenorphine | 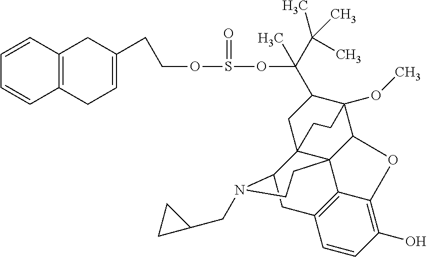 |
| diphenylcyclohexanol | phosphate triester | oxycodone | 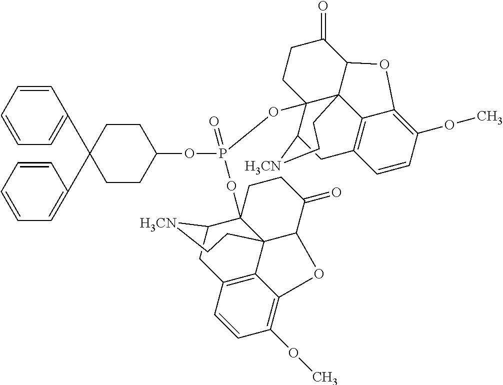 |
| diphenylcyclohexanol | phosphate triester | oxycodone naltrexone | 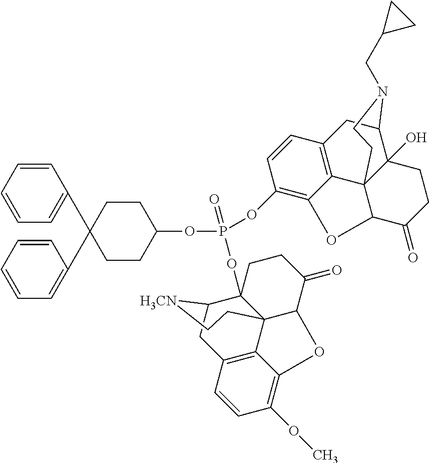 |
| diphenylcyclohexanol | succinate diester | (−)naltrexone | 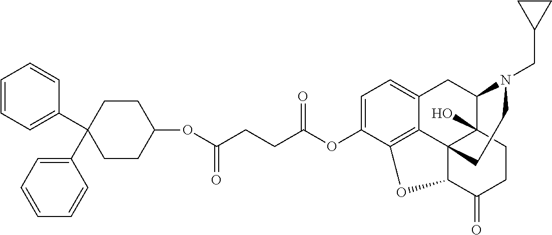 |
| diphenylcyclohexanol | succinate diester | (+)naltrexone | 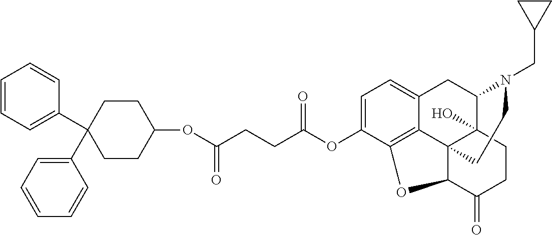 |

### EXAMPLES

The following example illustrates various aspects of the invention.

### Example 1

### Pharmacokinetic Assessment of Stabilized Opiate Composition

To assess the pharmacokinetics of a sustained-release opiate composition compared to the non-modified opiate, the following experiment may be conducted. Heparinized blood samples may be collected from a population of conscious rats (n=6) prior to the intravenous administration of codeine at a dose of 10 mg/kg to 50% of the rats (n=3) or codeine 4,4-diphenylcyclohexyl phosphate at a dose of 10 mg/kg to the other 50% of the rats (n=3). Heparinized blood samples may be collected via the femoral artery of all conscious rats at time intervals of 5 minutes, 30 minutes, 1 hour, 2 hours, 4 hours, 6 hours, 10 hours and 24 hours after the initial administration of the codeine compositions. All heparinized blood samples may be centrifuged after collection, and the resulting plasma may be collected and frozen.

All frozen plasma samples, as well as frozen samples of the codeine and codeine 4,4-diphenylcyclohexyl phosphate compositions administered to the rats, may be analyzed to determine the presence and concentrations of codeine in both free and bound forms. The measured plasma concentrations may be evaluated using a non-compartmental analysis method performed using existing analysis software (WinNonLin, Pharsight, St. Louis, Mo.) to determine terminal half lives and clearances. The terminal half life and clearance of the codeine and codeine 4,4-diphenylcyclohexyl phosphate may be statistically compared using a non-paired, two-tailed T-test. The terminal half life of the codeine 4,4-diphenylcyclohexyl phosphate may be determined to be significantly greater than the codeine composition, and the clearance of the codeine 4,4-diphenylcyclohexyl phosphate may be determined to be significantly smaller than the codeine composition at each sample time. These findings may indicate that the modification of codeine to codeine 4,4-diphenylcyclohexyl phosphate may result in a more sustained release form of codeine compared to unmodified codeine.

Having described the invention in detail, it will be apparent that modifications and variations are possible. Those of skill in the art should, in light of the present disclosure, appreciate that many changes could be made in the specific embodiments that are disclosed and still obtain a like or similar result without departing from the spirit and scope of the invention, therefore all matter set forth is to be interpreted as illustrative and not in a limiting sense.

What is claimed is:

1. A sustained-release opiate composition comprising:
a. an opiate;
b. a non-peptide blood albumin binder; and,
c. a connecting bridge attached to the opiate and to the non-peptide blood albumin binder.

2. The composition of claim 1, wherein the opiate is chosen from adulmine, allocryptopine, aporphine, benzylmorphine, berberine, bicuculine, bicucine, bulbocapnine, buprenorphine, butorphanol, canadine, capaurine, chelerythrine, chelidonine, codamine, codeine, coptisine, coreximine, corlumine, corybulbine, corycavamine, corycavine, corydaline, corydine, corytuberine, cularine, cotarnine, cryptopine, cycloartenol, cycloartenone, cyclolaudenol, dehydroreticuline, desomorphine, dextropropoxyphene, dextrorphanol, diacetylmorphine, dicentrine, dihydrosanguinarine, dipropanoylmorphine, epiporphyroxine, ethylmorphine, eupaverine, fagarine, fentanyl, glaucine, homochelidonoine, hydrocodone, hydrocotarnine, hydromorphone, hydroxythebaine, isoboldine, isocorybulbine, isocorydine, isocorypalmine, isoquinoline, laudanidine, laudanine, laudanosine, levorphanol, magnoflorine, meconic acid, methadone, morphine, nalbuphine, nalmefene, naloxone, naltrexamine, α-naltrexol, β-naltrexol, naltrexone, naphthaphenanthridine, narceine, narceinone, narcotoline, narcotine, neopine, nicomorphine, norlaudanosoline, norsanguinarine, noscapine, opium, oripavine, oxycodone, oxymorphone, oxysanguinarine, palaudine, papaverine, papaveraldine, papaverrubine, perparin, pethidine, phenanthrene, phtalide-isoquinoline, porphyroxine, protopine, pseudocodeine, pseudomorphine, reticuline, salutaridine, sinoacutine, sanguinarine, scoulerine, somniferine, stepholidine, tapentadol, tetrahydroprotoberberine, thebaine, tramadol, and xanthaline.

3. The composition of claim 1, wherein the non-peptide blood albumin binder comprises one or more functional groups chosen from aliphatic and aryl groups, wherein the aliphatic and aryl groups comprise between about 1 and 60 carbons and any one or more substituents chosen from a nitrogen, an oxygen, a sulfur, a halogen, an alkyl group, an amide, an ester, and a sulfonamide.

4. The composition of claim 1, wherein the connecting bridge comprises one or more functional groups chosen from phosphate diester, carbonate, and sulphonate.

5. The composition of claim 1, wherein:
a. the opiate is chosen from oxycodone, oxymorphone, hydrocodone, hydromorphone, buprenorphine, naloxone, and naltrexone;
b. the non-peptide blood albumin binder is chosen from diphenylcyclohexanol, phenylhexanol, and biphenylpropanol; and,
c. the connecting bridge is a phosphate diester.

6. The composition of claim 5, wherein the non-peptide blood albumin binder is diphenylcyclohexanol.

7. The composition of claim 1, wherein the composition further comprises a second opiate attached to the connecting bridge.

8. A sustained-release opiate composition comprising:
a. a first compound comprising a first opiate, a first non-peptide blood albumin binder, and a first connecting bridge attached to the first opiate and to the first non-peptide blood albumin binder; and,
b. a second compound comprising a second opiate, a second non-peptide blood albumin binder, and a second connecting bridge attached to the second opiate and to the second non-peptide blood albumin binder.

9. The composition of claim 8, wherein the first opiate is essentially the same substance as the second opiate, and wherein the first opiate and the second opiate are chosen from adulmine, allocryptopine, aporphine, benzylmorphine, berberine, bicuculine, bicucine, bulbocapnine, buprenorphine, butorphanol, canadine, capaurine, chelerythrine, chelidonine, codamine, codeine, coptisine, coreximine, corlumine, corybulbine, corycavamine, corycavine, corydaline, corydine, corytuberine, cularine, cotarnine, cryptopine, cycloartenol, cycloartenone, cyclolaudenol, dehydroreticuline, desomorphine, dextropropoxyphene, dextrorphanol, diacetylmorphine, dicentrine, dihydrosanguinarine, dipropanoylmorphine, epiporphyroxine, ethylmorphine, eupaverine, fagarine, fentanyl, glaucine, homochelidonoine, hydrocodone, hydrocotarnine, hydromorphone, hydroxythebaine, isoboldine, isocorybulbine, isocorydine, isocorypalmine, isoquinoline, laudanidine, laudanine, laudanosine, levorphanol, magnoflorine, meconic acid, methadone, morphine, nalbuphine, nalmefene, naloxone, naltrexamine, α-naltrexol, β-naltrexol, naltrexone, naphthaphenanthridine, narceine, narceinone, narcotoline, narcotine, neopine, nicomorphine, norlaudanosoline, norsanguinarine, noscapine, opium, oripavine, oxycodone, oxymorphone, oxysanguinarine, palaudine, papaverine, papaveraldine, papaverrubine, perparin, pethidine, phenanthrene, phtalide-isoquinoline, porphyroxine, protopine, pseudocodeine, pseudomorphine, reticuline, salutaridine, sinoacutine, sanguinarine, scoulerine, somniferine, stepholidine, tapentadol, tetrahydroprotoberberine, thebaine, tramadol, and xanthaline.

10. The composition of claim 8, wherein the first non-peptide blood albumin binder and the second non-peptide blood albumin binder are essentially the same substance chosen from diphenylcyclohexanol, phenylhexanol, and biphenylpropanol.

11. The composition of claim 8, wherein the first connecting bridge and the second connecting bridge comprise different substances chosen from phosphate diester, carbonate, and sulphonate.

12. The composition of claim 8, wherein the first connecting bridge and the second connecting bridge are cleaved at different times after administration of the composition.

13. The composition of claim 8, wherein the composition further comprises a third compound comprising a third opiate, a third non-peptide blood albumin binder and a third connecting bridge attached to the third opiate and to the third non-peptide blood albumin binder, wherein the third connecting bridge is a different substance from the first connecting bridge and the second connecting bridge, and wherein the third connecting bridge is selected from phosphate diester, carbonate, and sulphonate.

14. A sustained-release opiate composition comprising:
a. a first opiate;
b. a second opiate;
c. a non-peptide blood albumin binder; and,
d. a first connecting bridge attached to the first opiate, to the second opiate, and to the non-peptide blood albumin binder.

15. The composition of claim 14, wherein the first opiate is essentially the same substance as the second opiate, and wherein the first opiate and the second opiate are chosen from adulmine, allocryptopine, aporphine, benzylmorphine, berberine, bicuculine, bicucine, bulbocapnine, buprenorphine, butorphanol, canadine, capaurine, chelerythrine, chelidonine, codamine, codeine, coptisine, coreximine, corlumine, corybulbine, corycavamine, corycavine, corydaline, corydine, corytuberine, cularine, cotarnine, cryptopine, cycloartenol, cycloartenone, cyclolaudenol, dehydroreticuline, desomorphine, dextropropoxyphene, dextrorphanol, diacetylmorphine, dicentrine, dihydrosanguinarine, dipropanoylmorphine, epiporphyroxine, ethylmorphine, eupaverine, fagarine, fentanyl, glaucine, homochelidonoine, hydrocodone, hydrocotarnine, hydromorphone, hydroxythebaine, isoboldine, isocorybulbine, isocorydine, isocorypalmine, isoquinoline, laudanidine, laudanine, laudanosine, levorphanol, magnoflorine, meconic acid, methadone, morphine, nalbuphine, nalmefene, naloxone, naltrexamine, α-naltrexol, β-naltrexol, naltrexone, naphthaphenanthridine, narceine, narceinone, narcotoline, narcotine, neopine, nicomorphine, norlaudanosoline, norsanguinarine, noscapine, opium, oripavine, oxycodone, oxymorphone, oxysanguinarine, palaudine, papaverine, papaveraldine, papaverrubine, perparin, pethidine, phenanthrene, phtalide-isoquinoline, porphyroxine, protopine, pseudocodeine, pseudomorphine, reticuline, salutaridine, sinoacutine, sanguinarine, scoulerine, somniferine, stepholidine, tapentadol, tetrahydroprotoberberine, thebaine, tramadol, and xanthaline.

16. The composition of claim 14, wherein the non-peptide blood albumin binder comprises one or more functional groups chosen from aliphatic or aryl groups, wherein the aliphatic or aryl groups comprise between about 1 and 60 carbons and any one or more substituents chosen from a nitrogen, an oxygen, a sulfur, a halogen, an alkyl group, an amide, an ester, and a sulfonamide.

17. The composition of claim 14, wherein the connecting bridge comprises one or more functional groups chosen from phosphate diester, carbonate, and sulphonate.

18. The composition of claim 14, wherein:
a. the first opiate is selected from oxycodone, oxymorphone, hydrocodone, hydromorphone, buprenorphine, naloxone, and naltrexone;
b. the second opiate is essentially the same substance as the first opiate;
c. the non-peptide blood albumin binder is selected from diphenylcyclohexanol, phenylhexanol, and biphenylpropanol; and,
d. the connecting bridge is a phosphate diester.

19. The composition of claim 14, wherein the non-peptide blood albumin binder is diphenylcyclohexanol and the connecting bridge is phosphate diester.

20. The composition of claim 14, wherein the composition is administered at a dosage equal to or greater than a dosage recommended for the corresponding opiate administered without the non-peptide blood albumin binder and the connecting bridge.
